(19) United States
(12) Patent Application Publication
(10) Pub. No.: US 2025/0025563 A1
(43) Pub. Date: Jan. 23, 2025

(54) MODIFIED SOPHOROLIPID FOR IMPROVED BIOAVAILABILITY OF AMINO ACIDS AND METHOD OF USE

(71) Applicant: LOCUS SOLUTIONS IPCO, LLC, SOLON, OH (US)

(72) Inventors: KEN ALIBEK, SOLON, OH (US); LEE SPEIGHT, SOLON, OH (US); ANDREW MORRIS, RICHMOND, VA (US); DANIEL HAGAMAN, MECHANICSVILLE, VA (US); SEAN FARMER, FT. LAUDERDALE, FL (US)

(21) Appl. No.: 18/252,394

(22) PCT Filed: Apr. 6, 2023
(86) PCT No.: PCT/US2023/065419

§ 371 Date: May 10, 2023

Related U.S. Application Data
(60) Provisional application No. 63/328,883, filed on Apr. 8, 2022.

(51) Int. Cl. A61K 47/54

(52) U.S. Cl. CPC A61K 47/549

(57) ABSTRACT

The present invention provides modified sophorolipids modified with amino acids to improve, e.g., the bioavailability of the amino acids. The modified sophorolipids of the present invention are capable of efficiently delivering the amino acids to the target site and enhancing the efficacy of the amino acid and/or the sophorolipids.

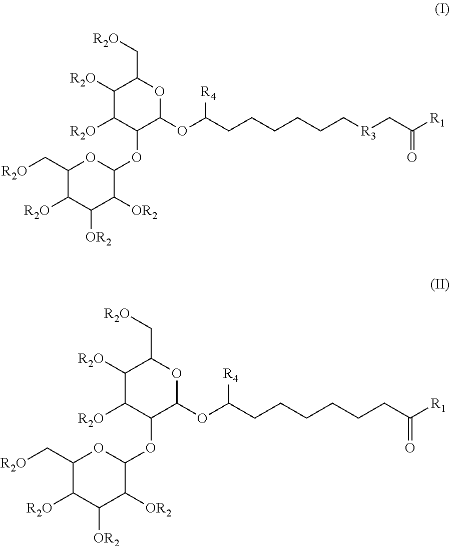

### CROSS-REFERENCE TO RELATED APPLICATION

This application claims priority to U.S. Provisional Patent Application No. 63/328,883, filed Apr. 8, 2022, which is incorporated herein by reference in its entirety.

### BACKGROUND OF THE INVENTION

Sophorolipids are classified as glycolipid biosurfactants as they are produced by fermentation of microbes, such as Starmerella bombicola, using carbohydrate and lipids as carbon sources. Sophorolipids are amphiphilic molecules containing a hydrophilic carbohydrate group (sophorose) and a hydrophobic fatty acid. Sophorose is a disaccharide that consists of two glucose molecules linked via a β-1,2 bond. The sophorose in sophorolipids can be acetylated, particularly at the 6′-and/or 6″-positions. The fatty acid in sophorolipids is β-glycosidically attached to the sophorose molecule through its hydroxyl group, and its terminal carboxylic acid group is either free (as shown in FIG. 1) or internally esterified (i.e., lactonic form), generally at the 4″-position. The fatty acid of sophorolipids generally has 16 or 18 carbon atoms with one unsaturation. However, it can consist of chain lengths from C12 to C22 having various degrees and positions of unsaturation. Thus, sophorolipids are frequently produced as a mixture of related molecules. Differences among the related molecules arise mainly from: their fatty acid structure (degree of unsaturation, chain length, position(s) of unsaturation and position of hydroxylation); whether they are produced in the free or lactonic form, the acetylation pattern; the presence of stereoisomers; and/or whether the β-glycosidic bond on the fatty acid is at the ω-position (e.g., terminal) or ω-1 position (sub-terminal).

Sophorolipids have many advantageous characteristics that make them superior to synthetic surfactants, such as biodegradability, low toxicity, high surface and interfacial activities, and stability in under wide ranges of temperatures, pressures, and ionic strengths.

In recent years, chemical modifications of sophorolipids have been pursued with an eye toward enhancing the properties of sophorolipids. An example is poly(sophorolipid), which is a polymer of sophorolipids having potential biomaterials applications.

### BRIEF SUMMARY OF THE INVENTION

One aspect of the present invention provides sophorolipids or “truncated” sophorolipids modified with amino acids. In some embodiments, the modified sophorolipids improve the bioavailability of the linked amino acids compared to free amino acids. In other embodiments, the modified sophorolipids enhance the efficacy of the sophorolipid and/or the amino acid.

According to a first set of embodiments, the present invention provides modified sophorolipids of the formula (I) or salts thereof:

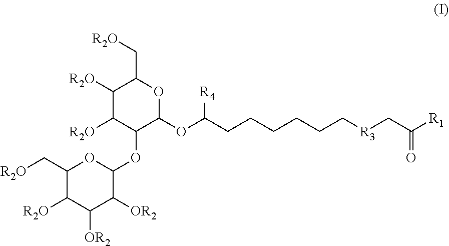

wherein:
R₁ is an amino acid linked to the adjacent carbonyl moiety of the sophorolipid structure via an amide linkage;
R₂ is each independently H or —COCH₃;
R₃ is a saturated or unsaturated aliphatic hydrocarbon chain having 5-15 carbons; and
R₄ is H or —CH₃.

According to a second set of embodiments, the present invention provides modified sophorolipids of the formula (II) or salts thereof:

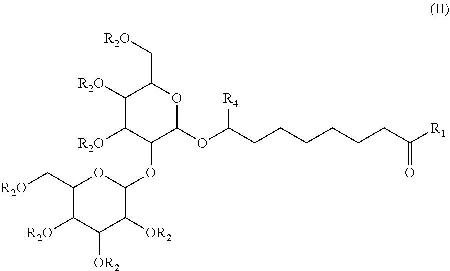

wherein:
R₁ is an amino acid linked to the adjacent carbonyl moiety of the sophorolipid structure via an amide linkage;
R₂ is each independently H or —COCH₃; and
R₄ is H or —CH₃.

The present invention encompasses all of the compounds represented by the general formulas (I) and (II), including salts thereof, hydrates thereof, geometric and optical isomers thereof, and polymorphic forms thereof.

Other aspects of the present invention are directed to a method of improving the bioavailability of amino acids. The modified sophorolipids of the present invention are capable of efficiently delivering and enhancing the efficacy of the linked amino acids by improving the bioavailability of the amino acids. In other embodiments, the modified sophorolipids enhance the efficacy of the sophorolipid as well as the amino acid.

Additionally, certain embodiments of the present invention provide a method of treating or preventing various diseases, disorders, and/or conditions by administering the modified sophorolipid.

Further embodiments of the present invention are directed to a method of improving nutrition in a subject's diet by administering the modified sophorolipid incorporating the amino acid(s) that is or may be lacking or insufficient in the subject's diet.

### BRIEF DESCRIPTION OF THE DRAWINGS

FIG. 1 illustrates an exemplary general structure of free acidic forms of sophorolipids, wherein: R is each independently H or acetyl (—COCH₃); R_{c} denotes a carbon chain, typically C10 to C20 in length, having various degrees and positions of unsaturation.

FIG. 2 illustrates an exemplary structure of α-amino acids.

FIG. 3 illustrates an exemplary structure of β-amino acids.

FIG. 4 illustrates an exemplary structure of γ-amino acids.

FIG. 5 illustrates an exemplary structure of δ-amino acids.

### DETAILED DESCRIPTION OF THE INVENTION

### Modified Sophorolipids

In one aspect, the present invention provides modified sophorolipids of the general formula (I), (II), or a salt thereof, in which a sophorolipid or a truncated sophorolipid is linked to an amino acid via an amide linkage. By “truncated,” it is meant that the carbon chain of the fatty acid is shorter than the carbon chain of the fatty acids normally present in sophorolipids.

According to a first set of embodiments, the present invention provides modified sophorolipids of the formula (I) or salts thereof:

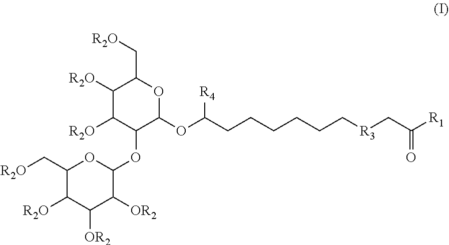

wherein:
R₁ is an amino acid linked to the adjacent carbonyl moiety of the sophorolipid structure via an amide linkage;
R₂ is each independently H or —COCH₃;
R₃ is a saturated or unsaturated aliphatic hydrocarbon chain having 5-15 carbons; and
R₄ is H or —CH₃.

In some preferred embodiments, the modified sophorolipids of the formula (I) or salts thereof have R₃ that is an aliphatic hydrocarbon chain of 8 carbons with one unsaturation in the form of a double bond, R₄ that is methyl (—CH₃), and all R₂ are H.

In further embodiments, the modified sophorolipids of the formula (I) or salts thereof have the following structure:

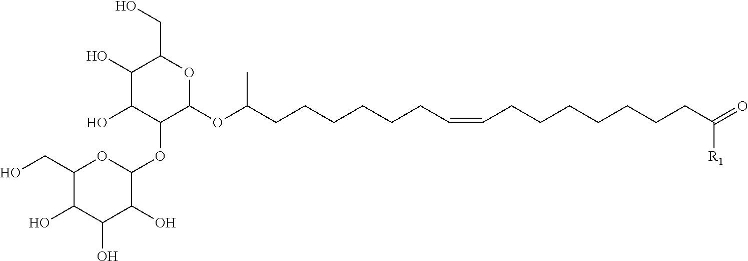

wherein R₁ is an amino acid linked to the carbonyl moiety of the sophorolipid structure via an amide linkage.

According to a second set of embodiments, the present invention provides modified sophorolipids of the formula (II) or salts thereof:

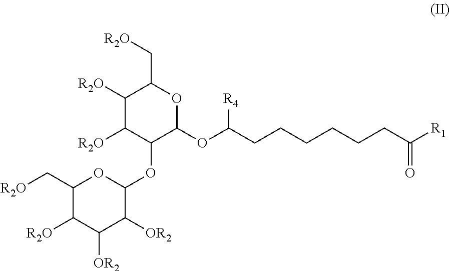

wherein:
R₁ is an amino acid linked to the adjacent carbonyl moiety of the sophorolipid structure via an amide linkage;
R₂ is each independently H or —COCH₃; and
R₄ is H or —CH₃.

In some preferred embodiments, the modified sophorolipids of the formula (II) or salts thereof have R₄ that is methyl (—CH₃) and R₂ that is all H.

In further embodiments, the modified sophorolipids of the formula (II) or salts thereof have the following structure:

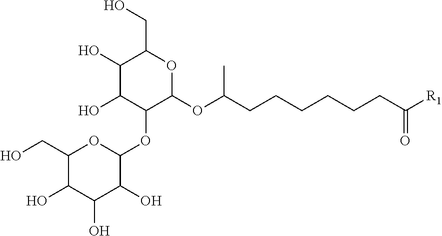

wherein R₁ is an amino acid linked to the carbonyl moiety of the sophorolipid structure via an amide linkage.

The present invention encompasses all of the compounds represented by the general formulas (I) and (II), including salts thereof, hydrates thereof, geometric and optical isomers thereof, and polymorphic forms thereof. Furthermore, it will be appreciated by a skilled artisan that the modified sophorolipid of formulas (I) and (II) may exist in lactonic forms if the amino acid of the R₁ group has a free acid such as carboxylic acid. That is, the free carboxylic acid can esterify internally with one of the hydroxyl substituents of the sophorose group.

In some embodiments, the modified sophorolipids improve the bioavailability of linked amino acids compared to free amino acids. In other embodiments, the modified sophorolipids enhance the efficacy of the sophorolipid and/or the amino acid.

### Sophorolipids

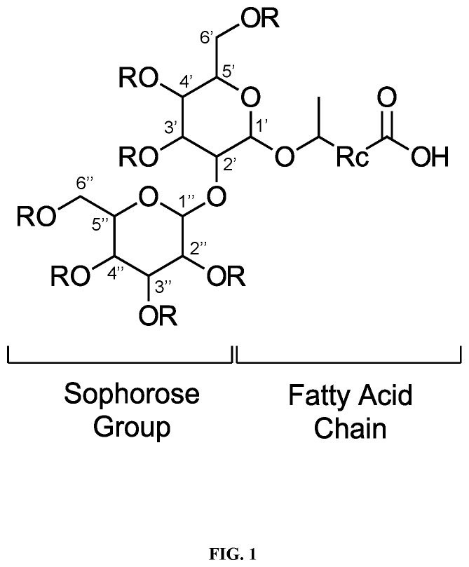

Sophorolipids are generally obtained from fermentations by microorganisms that use as carbon sources pure fatty acids, fatty acid mixtures, pure fatty acid esters, mixtures of fatty acid esters, triglycerides along with carbohydrate sources such as corn syrup, dextrins and glucose using a fermentation process comprising a wild-type or engineered yeast strain. As shown in FIG. 1, sophorolipids generally consist of a sophorose that may or may not be acetylated, particularly at the 6′ and 6″ positions, and a hydroxylated fatty acid tail with various degrees of unsaturation. Most commonly, the fatty acids consist of 16 or 18 carbon atoms with one degree of unsaturation, but the fatty acid structure (e.g., degree of unsaturation, chain length) varies depending on the carbon sources provided during the production and the microorganisms undergoing fermentation.

The production of sophorolipids with the use of renewable substrates and different microbial species, as well as the variation in culture parameters (incubation time, stirring speed, pH of the medium and added nutrients), allow for the acquisition of compounds with distinct structural and physical properties. This makes it possible to produce a wide variety of compounds that can elicit different physical, chemical, biochemical, and biophysical properties.

The hydrophobic fatty acid tail of sophorolipids normally is hydroxylated at the terminal and is β-glycosidically linked to the sophorose molecule. The fatty acid carboxylic acid group in sophorolipids is either free (acidic form as in FIG. 1) or intramolecularly esterified, generally at the 4″-position (lactonic form). The degree of lactonization of sophorolipids and acetylation of sophorose may be influenced by the carbon source fed during sophorolipid production. For example, it has been found that sophorolipids derived from rapeseed, sunflower and palm oils rich in C18:0 and C18:1 fatty acids are formed with higher levels of diacetylated lactones than sophorolipids produced from the corresponding fatty acid ester feedstocks.

Sophorolipids have environmental compatibility, high biodegradability, low toxicity, high selectivity, and specific activity in a broad range of temperature, pH, and salinity conditions. Suggested benefits of sophorolipids include, but are not limited to, killing of pathogenic agents in the skin, modulating the skin's immune system, killing melanocytes to allow for replacement cells to grow, reducing oxidative stress, enhancing multiplication and function of keratinocytes and fibroblasts, and enhancing dermal penetration of both the e.g., biosurfactants, and one or more other active ingredients in the composition. See, e.g., U.S. Publication No. 2020/0155444.

### Fermentation

In some embodiments, the sophorolipids modified with an amino acid according to the present invention are produced by a fermentation process known in the art in the presence of select fatty acids and carbohydrate feedstocks. In other embodiments, sophorolipids to be modified are first produced by a fermentation process known in the art, then chemically modified using known synthetic techniques to attach a desirable amino acid. As used herein “fermentation” refers to growth of cells under controlled conditions. The growth could be aerobic or anaerobic. The fermentation processes known in the art include, but are not limited to, solid-state fermentation, submerged fermentation, or modifications, hybrids and/or combinations thereof.

The modified sophorolipids of formula (I) or (II), or sophorolipids to be modified according to the present invention can be derived via a fermentation process from a recombinant organism or by a strain that naturally produces sophorolipids. Non-limiting examples of sophorolipid-producing organisms include Candida bombicola, Candida apicola, Candida bogoriensis, Yarrowia lipolytica, Starmerella bombicola, Starmerella clade, Rhodotorula bogoriensis, Wickerhamiella domericqiae, and Wickerhamomyces anomalus. Some recombinant sophorolipid-producing microbes have been reported to allow control of sophorolipid structure. As a non-limiting example, certain recombinant S. bombicola strains may be utilized to produce either solely lactonic or solely acidic sophorolipids (see Roelants et. al., Towards the Industrialization of New Biosurfactants: Biotechnological Opportunities for the Lactone Esterase Gene from Starmerella Bombicola, 113 Biotechnology and Bioengineering 3, 550-559 (2015)). As an additional example, a recombinant Candida bombicola strain with an acetyltransferase gene knockout can be used to produce sophorolipids without acetylation (see, e.g., WO 2012/080116).

The microorganisms of interest can be cultivated at small or large scale on site and utilized, even being still-mixed with their media. The modified sophorolipids of the present invention can also be produced at large quantities at the site of need. The growth vessel used for growing sophorolipid-producing organisms can be any fermenter or cultivation reactor for industrial use.

In one embodiment, a single type of microorganism is grown in a reactor system. In alternative embodiments, multiple microorganisms, which can be grown together without deleterious effects on growth or the resulting product, can be grown in a single reactor system. There may be, for example, 2 to 3 or more different microorganisms grown in a single reactor at the same time. In some embodiments, more than one microorganism grows symbiotically in the reactor.

In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

The cultivation may be supplemented with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, isopropyl, propanol, butanol, pentanol, hexanol, isobutanol, and/or glycerol; fats and oils such as soybean oil, rice bran oil, canola oil, olive oil, corn oil, sesame oil, and/or linseed oil; etc. These carbon sources may be used independently or in a combination of two or more.

In some embodiments, carbon sources utilized in the fermentation process already have been chemically modified to possess the desirable functional groups. For example, fatty acids can already be linked to an amino acid of choice before it is introduced to the reaction vessel containing sophorolipid-producing microorganisms. In other embodiments, fatty acids utilized in the fermentation process are not already linked to an amino acid.

The cultivation can be supplemented with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The growing culture in the cultivation can also be oxygenated. One embodiment utilizes slow motion of air to remove low oxygen-containing air and introduce oxygenated air. In the case of submerged fermentation, the oxygenated air may be ambient air supplemented daily through mechanisms including impellers for mechanical agitation of the liquid, and air spargers for supplying bubbles of gas to the liquid for dissolution of oxygen into the liquid.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included. Such amino acids include those that are not intended to be part of the modified sophorolipid of formula (I) or (II).

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride and/or sodium carbonate. These inorganic salts may be used independently or in a combination of two or more.

In some embodiments, when, for example, the microbes used to inoculate the substrate are in spore form (e.g., bacterial endospores), germination enhancers can be added to the substrate. Examples of germination enhancers according to the present invention include, but are not limited to, L-alanine, manganese, L-valine, and L-asparagine or any other known germination enhancer.

In some embodiments, additional acids and/or antimicrobials in the liquid medium before and/or during the cultivation process may be added. Antimicrobial agents or antibiotics are used for protecting the culture against contamination. Additionally, antifoaming agents may also be added to prevent the formation and/or accumulation of foam when gas is produced during cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the liquid medium may be necessary.

The method and equipment for cultivation of microorganisms and production of the microbial by-products can be performed in a batch, quasi-continuous, or continuous processes.

In one embodiment, the cultivation of microorganisms is carried out at about 5° C. to about 100° C., preferably, about 15° C. to about 60° C., more preferably, about 25° C. to about 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a microbe-free medium or contain cells, spores, mycelia, conidia, or other microbial propagules. In this manner, a quasi-continuous system is created.

In some embodiments, the modified sophorolipids of formula (I) or (II), or sophorolipids to be modified are produced by microorganisms of interest and retained in the microorganisms or secreted into their growth medium. The modified sophorolipid or sophorolipid to be modified can be, for example, at least 20%, 30%, 40%, 50%, 60%, 70%, 80%, or 90% w/v of the total removed composition.

The growth medium may contain compounds that stabilize the activity of the sophorolipids. The modified sophorolipid or sophorolipid to be modified can be purified, or such sophorolipids can be used in crude form, meaning they are not separated from the fermentation broth in which they were produced.

In certain embodiments, the modified sophorolipid or sophorolipid to be modified is isolated and/or purified from the growth medium resulting from fermentation of a biosurfactant-producing microorganism. Isolation and purification can be easily achieved using known methods or techniques described in the literature. The sophorolipid can be further concentrated, if desired.

As used herein, the terms “isolated” or “purified,” when used in connection with biological or natural materials such as glycolipids means the material is substantially free of other compounds, such as cellular material, with which it is associated in nature. That is, the materials do not occur naturally without these other compounds and/or have different or distinctive characteristics compared with those found in the native material.

In certain embodiments, purified compounds are at least 60% by weight of the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

### Chemical Modification Methods

In some embodiments, the modified sophorolipids according to the present invention are obtained by chemically modifying sophorolipids that have been produced by sophorolipid-producing microorganisms. The sophorolipids to be chemically modified may be obtained by fermentation methods as described above and/or any other methods known in the art, and then isolated or purified using known methods or techniques. Any suitable techniques and chemical reactions known in the art may be used to modify the resulting sophorolipids. For example, an amino acid may be linked to the sophorolipid structure via various reactions, including but are not limited to, amidation, reductive amination, and esterification. In preferred embodiments, an amino acid may be linked to the sophorolipid structure via amidation including but are not limited to amide coupling reaction.

Chemical modifications can also be made, for example, to alter the degree of unsaturation on the fatty acid chain. Commonly known hydrogenation or dehydrogenation reactions with or without a catalyst, or addition or elimination reactions may be used.

### Amino Acids

Modified sophorolipids of the present invention incorporates within their structure an amino acid linked to the fatty acid chain of the sophorolipid structure via an amide bond. In preferred embodiments, the amino acid is capable of being released from the sophorolipid structure during digestion thereof to impart its therapeutic, pharmaceutical, nutritional, or other benefits. By covalently attaching an amino acid to a sophorolipid or a truncated sophorolipid via an amide bond (i.e., formulas (I) and (II), respectively), the amino acid can be delivered, released, and absorbed by a subject's body, more efficiently than when the amino acid alone is administered to the subject.

Amino acid as used herein has at least one amino group and at least one acid such as carboxyl group in its backbone, along with a side chain that is different for each amino acid. The amino group in the backbone is capable of forming an amide linkage with the carbonyl moiety of the sophorolipids structure of formulas (I) and (II). The side chain can be a variety of substituents, including but are not limited to, hydrogen (e.g., as in glycine), organic, semi-organic or inorganic, and a ring that is cyclized on to the amine (e.g., as in proline). If a suitable amino group is present in the side chain, it may also form an amide linkage with the carbonyl moiety of the sophorolipids structure of formulas (I) and (II). The amino group and the acid (e.g., the carboxyl group) of the backbone may be attached to the same carbon (α-carbon) or different carbons.

It is further within the spirit and scope of the present invention for the amino acid to contain an acidic group other than carboxyl group in its backbone. For example, a sulfonic acid or a thiocarboxylic acid (i.e., wherein one of the oxygen atoms is replaced with a sulfur atom) may be present in the amino acid structure in lieu of the carboxyl group. A non-limiting example of such amino acid is taurine (also called 2-aminoethanesulfonic acid), which contains sulfonic acid and an amino group that is attached to the β-carbon.

The amino acid as used herein may be L-amino acid or D-amino acid. It can be natural amino acids (such as essential amino acids, non-essential amino acids, or conditional amino acids) or synthetic/modified amino acids that do not exist in nature. The present invention encompasses amino acids that are both incorporated into proteins and not incorporated into proteins. Amino acids that exist in nature may be purified products or can be artificially synthesized.

In aqueous solutions, amino acids—especially the common natural forms of amino acids—exist as zwitterions, wherein both the amino group and the acid (e.g., carboxyl group) are in charged states, for example, —NH₃⁺ or —NH₂⁺, —and —CO₂⁻. Depending on the pH of the environment, certain side chains may exist in charged states as well.

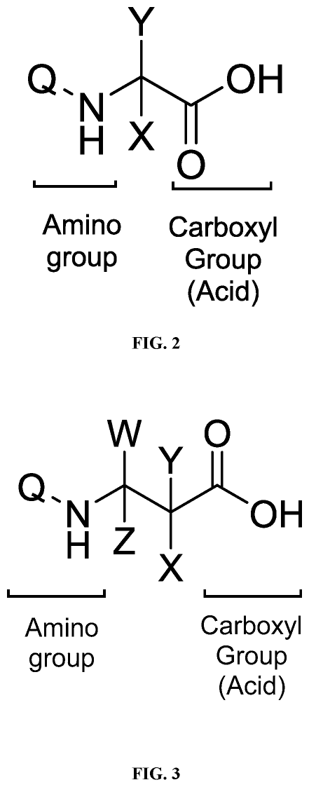

In some embodiments, the amino acid according to the present invention is an alpha (α)-amino acid. An exemplary structure is depicted in FIG. 2, which contains an amino group and a carboxyl group attached to the same carbon adjacent to the carboxyl group designated as the α-carbon. Also attached to the α-carbon are substituents X and Y, which may be same or different, and at least one of which is typically H. The amino group may be a primary amine wherein Q is H or substituted wherein Q is a substituent. Non-limiting examples of α-amino acids include glycine, methionine, lysine, and glutamine.

In other embodiments, the amino acid according to the present invention is a beta (β)-amino acid. An exemplary structure is depicted in FIG. 3, which contains an amino group attached to the β-carbon (second carbon from the carboxyl group). X, Y, W, and Z may be any substituent, which may be same or different, and at least one of which typically being H. The amino group may be a primary amine wherein Q is H or substituted wherein Q is a substituent. Non-limiting examples of β-amino acids include β-alanine and taurine.

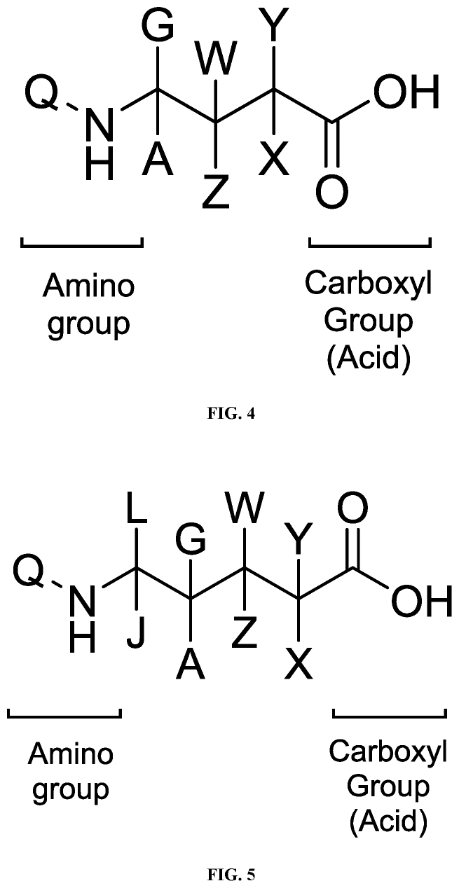

In yet other embodiments, the amino acid according to the present invention is a gamma (γ)-amino acid. An exemplary structure is depicted in FIG. 4, which contains an amino group attached to the γ-carbon (third carbon from the carboxyl group). X, Y, W, Z, A, and G may be any substituent, which may be same or different, and at least one of which typically being H. The amino group may be a primary amine wherein Q is H or substituted wherein Q is a substituent. Non-limiting examples of γ-amino acids include vigabatrin (4-amino-5-hexenoic acid) and gamma-Aminobutyric acid (GABA).

In further embodiments, the amino acid according to the present invention is a delta (δ)-amino acid. An exemplary structure is depicted in FIG. 5, which contains an amino group attached to the δ-carbon (fourth carbon from the carboxyl group). X, Y, W, Z, A, G, J, and L may be any substituent, which may be same or different, and at least one of which typically being H. The amino group may be a primary amine wherein Q is H or substituted wherein Q is a substituent. Non-limiting examples of δ-amino acids include 5-aminopentanoic acid and 5-amino-2-oxopentanoic acid.

A skilled artisan would appreciate that the general structures of amino acids may be further modified without departing from the spirit and scope of the invention. For example, the carbon chain of the backbone between the amino group and the acid may be longer than that of the δ-amino acids described above, or the acid may be esterified.

In some embodiments, the amino acid incorporated in the modified sophorolipids according to the invention is methionine (Met), lysine (Lys), or glutamine (Gln). In specific embodiments, these modified sophorolipids function to reduce factors contributing to stress in a subject, for example, reactive oxygen species (ROS) and cortisol.

Methionine (Met) is an essential amino acid and critical precursor to the cellular methyl donor S-adenosylmethionine. Methionine residues in proteins react readily with a variety of reactive oxygen species (ROS), scavenging the reactive species. After the reaction methionine forms methionine sulfoxide which can be reduced back to methionine intracellularly. Thus, methionine residues may act as catalytic antioxidants protecting both the protein where they are located and other macromolecules, including the cells themselves. Methionine is further thought to provide health benefits including aiding in the absorption of nutrients such as selenium and zinc, facilitating the excretion of heavy metals, preventing excess fat buildup in the liver, lowering cholesterol levels by increasing lecithin production in the liver. Methionine is also shown to be effective in the treatment of Tylenol (Acetaminophen) poisoning.

Lysine (Lys) is an essential amino acid and has been shown to act as a partial antagonist for partial serotonin receptor 4 (5-HT₄), decreasing blood levels of cortisol (a so called “stress hormone”) as well as stress-induced brain-gut response. In experimental animals, prolonged inadequacy of dietary lysine increases stress-induced anxiety. Lysine supplementation in humans with low dietary intake of L-lysine has been shown to reduce anxiety. In one study conducted on otherwise healthy adult humans, oral treatment with a combination of L-lysine and L-arginine reduced both trait anxiety and stress-induced state anxiety. Additionally, the combination decreased basal levels of salivary cortisol and chromogranin-A (a salivary marker of the sympatho-adrenal system) in male subjects. See, e.g., Smigra, M. et al., Oral Treatment with L-lysine and L-arginine Reduces Anxiety and Basal Cortisol Levels in Healthy Humans, Biomed Res. 28(2), 85-90 (2007). Thus, lysine supplementation has been suggested to be an effective reducer of anxiety and stress. Lysine supplementation has also been implicated in various other health benefits including suppression of cold sores, calcium absorption and retention, and wound healing.

Glutamine (Gln) is a conditional amino acid. This means that glutamine is capable of being synthesized by the body under normal physiological conditions, but under pathological conditions, the increasing demands from the body cause a relative lack of glutamine. Glutamine has anti-inflammatory and antioxidant properties, which make it a useful supplement for subjects engaged in intense physical activities in a high-stress environment such as athletes. Insufficient glutamine level leads to a disturbance in energy metabolism, immune suppression, and increased oxidative stress.

In other embodiments, the amino acid incorporated in the modified sophorolipids according to the invention is taurine. Taurine is a conditionally essential amino acid that is known to have antioxidant properties and serves a number of physiological functions in the body. Non-exhaustive examples of taurine's functions are discussed below. The modified sophorolipids incorporating taurine can be used for treating various ailments and/or maintaining general well-being.

First, various studies suggest that taurine supplementation can retard the initiation and progression of atherosclerosis and/or exerts preventive effects on other cardiovascular disorders and diseases. For example, decrease in cholesterol levels and inflammation have been observed in patients with heart failure. Ahmadian, M., Taurine Supplementation Has Anti-atherogenic and Anti-inflammatory Effects Before and After Incremental Exercise in Heart Failure, Ther. Adv. Cardiovasc. Dis. 11(7): 185-194 (2017). Moreover, taurine supplementation resulted in an antihypertensive effect in prehypertension and a significant improvement in endothelium-dependent and endothelium-independent vasodilation. Sun, Q. et al., Taurine Supplementation Lowers Blood Pressure and Improves Vascular Function in Prehypertension, Hypertension 67, 541-549 (2016). Combined with its anti-inflammatory effects, taurine's ability to lower blood pressure makes it a viable protective agent against coronary heart disease.

Taurine is present in high levels in mammalian skeletal muscles as well. Taurine is thought to control muscle metabolism and gene expression. Changes in the intracellular taurine level in skeletal muscle have been linked to different pathophysiological conditions, such as disuse-induced muscle atrophy, muscular dystrophy and/or senescence. Taurine supplementation has therapeutic potential to restore skeletal muscle function and performance. Furthermore, taurine treatment can be beneficial to reduce sarcolemmal hyper-excitability in myotonia-related syndromes. De Luca, A., Taurine: the appeal of a safe amino acid for skeletal muscle disorders, J. Transl. Med. 13, 243 (2015). Taurine supplementation may also improve exercise performance, for example, improving the time performance in long-distance running.

Similarly, taurine has been found to induce expression of connective tissue growth factor (CTGF) in osteoblasts, suggesting taurine's role in the connective tissue strength.

Additional benefits of taurine supplementation concern the central and peripheral nervous systems. taurine is required for development and functioning of the central and peripheral nervous system where it exerts osmoregulatory, neuromodulatory and anti-apoptotic actions. Taurine deficiency can lead to several neurologic deficits and sensory losses. Animal studies have shown that taurine helps alleviate symptoms of neurotoxicity and neurological impairment in rodents. It has also been suggested that taurine may improve or protect brain functions, in particular, learning and memory.

Taurine may also be used as a treatment for eye disorders such as glaucoma and diabetic retinopathy as well as for diabetes to reduce glucose levels and insulin resistance.

Other non-limiting examples of amino acids that can be used in the present invention include, but are not limited to, alanine, β-alanine, arginine, asparagine, aspartic acid, cysteine, homocysteine, glutamic acid, glycine, histidine, homotaurine, isoleucine, leucine, phenylalanine, proline, serine, threonine, tryptophan, tyrosine, valine, theanine, D-2-amino-3-guanidinopropionic acid, GABA, citrulline, tranexamic acid, aminocaproic acid, 4-amino-5-hexenoic acid, 4-oxaproline, 4-thioproline, 2-azaproline, 4-hydroxyproline, 1,5-disubstituted tetrazole, 2-amino isobutyric acid, sarcosine, 1-aminocyclopentane-1-carboxylic acid, beta alanine, 2-amino-cyclopentane carboxylic acid (beta-proline), 5-hydroxylysine, hydroxylysine-5-sulfate, hydroxylysine-5-nitrate, hydroxylysine-5-phosphate, serine-3-sulfate, threonine-3-sulfate, serine-3-nitrate, threonine-3-nitrate, serine-3-phosphate, threonine-3-phosphate, 2-hydroxy alkanoic acid, 5-aminopentanoic acid, and 5-amino-2-oxopentanoic acid.

Administration of modified sophorolipids according to the present invention provide a range of benefits depending on the amino acid being incorporated therein. Various amino acids regulate glucose and lipid metabolism and energy balance, increase mitochondrial biogenesis, and maintain immune homeostasis. Dietary amino acids are also major fuels for the small intestinal mucosa, as well as important substrates for syntheses of intestinal proteins, nitric oxide, polyamines, and other products of biological importance. Studies support therapeutic roles for amino acids including glutamine, glutamate, arginine, glycine, lysine, threonine, and/or sulfur-containing amino acids in gut-related diseases. For example, ingestion of glutamine has been shown to improve gut health by supporting the gut microbiome, gut mucosal wall integrity, and by modulating inflammatory responses.

### Pharmaceutically or Cosmetically Acceptable Salts

In some embodiments, the modified sophorolipids of the present invention are provided in the form of a pharmaceutically or cosmetically acceptable salt. In specific embodiments, the acid group of the amino acids incorporated in the modified sophorolipids may be deprotonated to form a salt. The side chains of amino acids may also form a corresponding salt if it contains an appropriate functional group. Salts may be formed by procedures well known and described in the art.

Examples of pharmaceutically acceptable cations for salts include, without limitation, aluminium, arginine, benzathine, calcium, chloroprocaine, choline, diethanolamine, ethanolamine, ethylenediamine, lysine, magnesium, histidine, lithium, meglumine, potassium, procaine, sodium, triethanolamine, and zinc. Preferred salts are lithium (Li⁺), sodium (Na⁺), and potassium (K⁺) salts of the amino acid.

Depending on the chemical characteristics of the amino acids in the modified sophorolipids, acid addition salts may be formed. Examples of pharmaceutically acceptable addition salts include, but are not limited to, the non-toxic inorganic and organic acid addition salts such as the acetate derived from acetic acid, the aconate derived from aconitic acid, the ascorbate derived from ascorbic acid, the benzenesulfonate derived from benzenesulfonic acid, the benzoate derived from benzoic acid, the cinnamate derived from cinnamic acid, the citrate derived from citric acid, the embonate derived from embonic acid, the enantate derived from enanthic acid, the formate derived from formic acid, the fumarate derived from fumaric acid, the glutamate derived from glutamic acid, the glycolate derived from glycolic acid, the hydrochloride derived from hydrochloric acid, the hydrobromide derived from hydrobromic acid, the lactate derived from lactic acid, the maleate derived from maleic acid, the malonate derived from malonic acid, the mandelate derived from mandelic acid, the methanesulfonate derived from methane sulphonic acid, the naphthalene-2-sulphonate derived from naphtalene-2-sulphonic acid, the nitrate derived from nitric acid, the perchlorate derived from perchloric acid, the phosphate derived from phosphoric acid, the phthalate derived from phthalic acid, the salicylate derived from salicylic acid, the sorbate derived from sorbic acid, the stearate derived from stearic acid, the succinate derived from succinic acid, the sulphate derived from sulphuric acid, the tartrate derived from tartaric acid, the toluene-p-sulphonate derived from p-toluene sulphonic acid, and the like.

Other salts which may not be considered pharmaceutically acceptable, may be useful in the preparation of salts useful as intermediates in obtaining a modified sophorolipid of the invention and its pharmaceutically acceptable salt.

In another embodiment, the modified sophorolipids of the invention are used in their respective acid form according to the present invention.

The modified sophorolipids of the invention may be provided in unsolvated or solvated forms together with a pharmaceutically acceptable solvent(s) such as water, ethanol, and the like. Solvated forms may also include hydrated forms such as the monohydrate, the dihydrate, the hemihydrate, the trihydrate, the tetrahydrate, and the like.

### Compositions

Another aspect of the invention is directed to compositions comprising the modified sophorolipid of the present invention, administered to a subject. Such compositions may be cosmetic, pharmaceutical, dietary, nutraceutical, or the like. In some embodiments, the modified sophorolipids according to the present invention serve as active ingredients in the composition.

The term “subject” or “patient,” as used herein, describes an organism, including mammals such as primates. Mammalian species that can benefit from the disclosed methods of treatment include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters.

The particular mode of administration as well as the dosage regimen designed to deliver an effective amount of the modified sophorolipids are appropriately decided, taking into account factors such as the nature of the amino acid, age, sex, and particulars of the subject, as well as the condition, the disease, and the disease state involved, and whether the purpose is preventative. As used herein, the terms “effective amount,” and “effective dose” are used to refer to an amount of something (e.g., a compound, a composition, time) is capable of causing a desired outcome (e.g., reducing cortisol levels, lowering blood pressure). Administration of an effective amount of the modified sophorolipids may be accomplished in daily or multi-daily doses of the modified sophorolipids over a period of a few days to months, or even years. If the modified sophorolipids is administered with another therapeutic agent, the effective amount of the modified sophorolipids may carry the amino acid in the same range as is typical for use of that amino acid as a monotherapy, or the amount may be lower than a typical monotherapy amount especially if the combination therapy results in a synergy.

The composition according to the present invention may comprise an isolated and/or purified form of the modified sophorolipid as an active ingredient. In other embodiments, the modified sophorolipids in a composition according to the present invention are provided with the fermentation broth in which they were produced. Such “crude form” can comprise, for example, at least 20%, 30%, 40%, 50%, 60%, 70%, 80% or 90% w/v of the modified sophorolipids in the broth.

The composition may further include additional active or inactive ingredients, suitable for the mode of administration and intended purpose, provided that such addition does not adversely interfere with the functions of the modified sophorolipids.

In yet other embodiments, acceptable carriers may be included in the composition of the present invention, depending on the form of the composition and/or mode of administration. Pharmaceutically or cosmetically acceptable carries include but are not limited to, pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid, or talc. If desired or suitable, a coating material may also be used such as glyceryl monostearate or glyceryl distearate, for example, to delay absorption in the gastrointestinal tract if appropriate and the pharmaceutical composition is in the form of a solid form.

In certain embodiments, the composition according to the present invention further comprises microbial growth by-products. In certain embodiments, the microbial growth by-products are amphiphilic molecules, enzymes and/or proteins. In one embodiment, the microbial growth by-products have antimicrobial and/or anti-biofilm properties.

In certain embodiments, the composition further comprises one or more amphiphilic molecules, wherein the amphiphilic molecules are biosurfactants selected from, for example, low molecular weight glycolipids (e.g., sophorolipids, rhamnolipids, mannosylerythritol lipids and trehalose lipids), lipopeptides (e.g., surfactin, iturin, fengycin, athrofactin and lichenysin), cellobiose lipids, flavolipids, phospholipids (e.g., cardiolipins), and high molecular weight polymers such as lipoproteins, lipopolysaccharide-protein complexes, and polysaccharide-protein-fatty acid complexes. The amphiphilic molecules may be a modified form, derivative, fraction, isoform, isomer, or subtype of a biosurfactant, including forms that are biologically or synthetically modified. In one embodiment, the one or more biosurfactants are present in the composition in critical micelle concentration (CMC).

The composition can be placed in containers of appropriate size, taking into consideration, for example, the intended use, the contemplated method of application, the size of the fermentation vessel, and any mode of transportation from microbe growth facility to the location of use. Thus, the containers into which the composition is placed may be, for example, from 0.1 gallon to 1,000 gallons or more. The composition may further be placed into smaller containers, such as bottles, for distribution of individual doses of the composition.

The composition according to the present invention may come in various physical forms. In some embodiments, the composition is in the form of solids including tablets, filled capsules, powder and pellet forms. In another embodiment, the pharmaceutical composition may be in the powder form, in which the pharmaceutically accepted carrier is a finely divided solid that is in a mixture with the finely divided active ingredient, i.e., the modified sophorolipids. In a further embodiment, the pharmaceutical composition according to the present invention is a sustained release system such as semipermeable matrices of solid hydrophobic polymers containing the modified sophorolipids of the present invention. In another embodiment, the pharmaceutical composition is in a liquid form such as aqueous or non-aqueous solutions, suspensions, emulsions, elixirs, and capsules filled with the same.

Any appropriate mode of administration can be utilized to administer the compositions comprising the modified sophorolipids according to the present invention. Such mode includes, but is not limited to, oral, rectal, bronchial, nasal, topical, buccal, sub-lingual, transdermal, vaginal, intramuscular, intraperitoneal, intravenous, intra-arterial, subcutaneous, intracerebral, intraocular administration or in a form suitable for administration by inhalation or insufflation, including powders and liquid aerosol administration, or intraparenteral infusion. Administration may be also by way of other carriers or vehicles such as patches, micelles, liposomes, vesicles, implants (e.g., microimplants), synthetic polymers, and the like. Factors that determine the mode of administration of a particular composition include the nature of the amino acid that is being delivered to the subject. In some embodiments, the mode of administration is oral. In other embodiments, the mode of administration is rectal.

Orally administered compositions according to the invention are any preparations or compositions suitable for consumption, for nutrition, for oral hygiene and/or for pleasure, and are products intended to be introduced into the human or animal oral cavity, to remain there for a certain period of time and then to either be swallowed (e.g., food ready for consumption) or to be removed from the oral cavity again (e.g., chewing gums or products of oral hygiene or medical mouth washes). These products include all substances or products intended to be ingested by humans or animals in a processed, semi-processed or unprocessed state. This also includes substances that are added to orally consumable products (particularly food and pharmaceutical products) during their production, treatment or processing and intended to be introduced into the human or animal oral cavity.

In certain embodiments, the composition can further comprise components that are, for example, sources of energy, nutrients and/or other health-promoting supplements, flavorings, preservatives, pH adjusters, sweeteners and/or dyes. The composition may also be carbonated. In one embodiment, the composition is formulated as a dehydrated powder or concentrate that can be reconstituted into a drinkable fluid by the addition of water. In one embodiment, the composition is formulated as a blended smoothie or milkshake.

Orally administered compositions can also include substances intended to be swallowed by humans or animals and then digested in an unmodified, prepared, or processed state; the orally administered compositions according to the invention therefore also include casings, coatings or other encapsulations that are intended also to be swallowed together with the product or for which swallowing is to be anticipated.

In other embodiments, the compositions according to the present invention are formulated for direct administration into the GI tract. For example, the composition can be formulated for administration to the proximal lower GI by hand or colonoscopy, to the distal lower GI tract via enema or rectal tubes, and the upper GI tract via nasogastric tubes, duodenal tubes, and endoscopy/gastroscopy.

In certain embodiments, the present invention provides topical skin care compositions comprising one or more of the modified sophorolipids according to the present invention. In some embodiments, the topical composition can further comprise a dermatologically acceptable carrier, and one or more active or inactive cosmetic ingredients, such as, e.g., vitamins, moisturizers, dyes, fragrances, sunscreens, exfoliants, essential oils, botanical extracts, and so on.

The dermatologically acceptable carriers may include, for example, water; saline; physiological saline; ointments; creams; oil-water emulsions; water-in-oil emulsions; silicone-in-water emulsions; water-in-silicone emulsions; wax-in-water emulsions; water-oil-water triple emulsions; microemulsions; gels; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane and isohexadecane; silicone oils such as cyclomethicone, dimethicone, dimethicone cross-polymer, polysiloxanes and their derivatives, preferably organo-modified derivatives including PDMS, dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous carriers may include one or more solvents miscible with water, including lower alcohols, such as ethanol, isopropanol, and the like. The carrier may comprise from about 1% to about 99% by weight of the total composition, from 10% to about 85%, from 25% to 75%, or from 50% to about 65%.

Non-biological surfactants can also be added to the formulation. Examples of surfactants include, but are not limited to, alkyl sulfates, alkyl ether sulfates (e.g., sodium/ammonium lauryl sulfates and sodium/ammonium laureth sulfates), amphoterics (e.g., amphoacetates and amphopropionates), sulfosuccinates, alkyl polyglucosides, betaines (e.g., cocamidopropyl betaine), sultaines, sacrosinates, isethionates, taurates, ethoxylated sorbitan esters, alkanolamides and amino acid-based surfactants.

Viscosity modifiers can also be added to the compositions, including, for example, cocamide DEA, oleamide DEA, sodium chloride, cellulosic polymers, polyacrylates, ethoxylated esters, alcohol, glycols, xylene sulfonates, polysorbate 20, alkanolamides, and cellulose derivatives (e.g., hydroxypropyl methylcellulose and hydroxyethyl cellulose).

Polymers can also be added, including, for example, xanthan gum, guar gum, polyquaternium-10, PEG-120 methyl glucose dioleate, PEG-150 distearate, PEG-150 polyglyceryl-2 tristearate and PEG-150 pentaerythrityl tetrastearate.

In one embodiment, the composition may include additional skin actives, including but not limited to, keratolytic agents, desquamating agents, keratinocyte proliferation enhancers, collagenase inhibitors, elastase inhibitors, depigmenting agents, anti-inflammatory agents, steroids, anti-acne agents, antioxidants (e.g., ascorbic acid), advanced glycation end-product (AGE) inhibitors, exfoliating agents (e.g., glycolic acid, 3,6,9-trioxaundecanedioic acid, etc.), estrogen synthetase stimulating compounds (e.g., caffeine and derivatives), compounds capable of inhibiting 5 alpha-reductase activity (e.g., linolenic acid, linoleic acid, finasteride, and mixtures thereof), barrier function enhancing agents (e.g., ceramides, glycerides, cholesterol and its esters, alpha-hydroxy and omega-hydroxy fatty acids and esters thereof), retinoids, sunscreen, to name a few.

The composition may optionally comprise additives, adjuvants, or other components of topical compositions known to those skilled in the art including, but not limited to: skin penetration enhancers; emollients (e.g., isopropyl myristate, petrolatum, volatile or non-volatile silicones oils, such as methicone and dimethicone, ester oils, mineral oils, and fatty acid esters); humectants (e.g., glycerin, hexylene glycol, caprylyl glycol); skin plumpers (e.g., palmitoyl oligopeptide, collagen, collagen and/or glycosaminoglycan (GAG) enhancing agents); anti-inflammatory agents (e.g., Aloe vera, bioflavonoids, diclofenac, salicylic acid); chelating agents (e.g., EDTA or a salt thereof, such as disodium EDTA); vitamins (e.g., tocopherol and ascorbic acid); vitamin derivatives (e.g., ascorbyl monopalmitate, tocopheryl acetate, Vitamin E palmitate); thickeners (e.g., hydroxyalkyl cellulose, carboxymethylcellulose, carbombers, and vegetable gums, such as xanthan gum); gelling agents (e.g., ester-terminated polyester amides); structuring agents; proteins (e.g., lactoferrin); immune modulators (e.g., corticosteroids and non-steroidal immune modulators). Further examples include, but are not limited to: stabilizers; fragrances; film formers; insect repellents; skin cooling compounds; skin protectants; lubricants; preservatives; pearls; chromalites; micas; conditioners; anti-allergenics; pH adjusters; and antimicrobials.

The composition may be formulated as a suspension, emulsion, hydrogel, multiphase solution, vesicular dispersion or in any other known form of topical skin composition.

In certain embodiments, the topical composition may be formulated so that it can be applied, for example, via pen, tube, bottle, brush, stick, sponge, cotton swab, towelette (wipe), sprayer, dropper, hand, or finger.

The topical composition may be formulated in a variety of product forms, such as, for example, a lotion, cream, serum, spray, aerosol, liquid cake, ointment, essence, gel, paste, patch, pencil, powder, towelette, soap or other cleanser, shampoo, conditioner, stick, foam, mousse, elixir or concentrate.

In one embodiment, the composition can be provided in the form of a facial mask. The mask may be a peel-off mask or wash-off mask comprising the composition of the present invention.

In other embodiments, the composition of the present invention can be provided in a wound dressing, bandage, or a patch that may be applied, attached, or coupled to one or more layers of the skin or tissue of the subject. For example, the composition may be applied to a wound dressing, bandage, or a patch, which can then be placed over the area of skin being treated.

A wound dressing, bandage, and a patch can be made, for example, from any material that is dermatologically acceptable and suitable for placing on a wound or the skin. In exemplary embodiments, the wound dressing, bandage, and a patch may be made from a woven or non-woven fabric of synthetic or non-synthetic fibers, or any combination thereof. The dressing may also include a support, such as a polymer foam, a natural or man-made sponge, a gel or a membrane that may absorb or have disposed thereon, a composition. By way of example, the support can be a film, a natural or synthetic polymer, or a rigid or malleable material (e.g., gauze). The wound dressing, bandage, and a patch may be absorbent and can be, for example, wetted with a composition of the present invention before applying the gauze to a wound, scar or other sites.

In one embodiment, the wound dressing, bandage, or a patch may be impregnated with a composition of the subject invention and, optionally, dried. This allows the impregnated material to be stored for later use, or to avoid excessively dampening of the skin.

In other embodiments, the pharmaceutical composition may be formulated for parenteral administration (e.g., by injection, for example bolus injection or continuous infusion). In addition, the composition may be presented in unit dose form in ampoules, pre-filled syringes, and small volume infusion or in multi-dose containers with or without an added preservative. The composition may be in forms of suspensions, solutions, or emulsions in oily or aqueous carriers. The composition may further contain formulation agents such as suspending, stabilizing and/or dispersing agents. In a further embodiment, the active ingredient of the composition according to the invention may be in a powder form, obtained by aseptic isolation of sterile solid or by lyophilization from solution for constitution with a suitable carrier, e.g., sterile, pyrogen-free water, before use.

In yet other embodiments, the pharmaceutical composition may be formulated for direct administration to the nasal cavity by conventional means, for example with a dropper, pipette, or spray. The compositions may be provided in single or multi-dose form. Administration to the respiratory tract may also be achieved by means of an aerosol formulation in which the active ingredient is provided in a pressurized pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin.

### Methods of Improving Bioavailability, Physiological Functions, and Nutrition

One aspect of the present invention is directed to a method of improving the bioavailability of amino acids in a subject, preferably oral bioavailability. The method comprises providing a modified sophorolipid of formula (I) and/or (II), or a salt thereof as described herein wherein R₁ is an amino acid linked to the carbonyl moiety of the sophorolipid structure via an amide linkage. In particular, the modified sophorolipids of the present invention are capable of carrying one amino acid per molecule, wherein the amino acid is linked to the carbonyl moiety of a sophorolipid structure via an amide bond. A suitable amino acid has a chemical structure described herein with at least one amino functional group that is capable of forming an amide linkage with the carbonyl moiety at the defined attachment points of formulas (I) and (II).

According to certain embodiments of the invention, the modified sophorolipids are able to enhance and amplify the beneficial effects of the linked amino acid. Specifically, the modified sophorolipids of formulas (I) and (II) or a salt thereof incorporating an amino acid contributes to more efficient and successful absorption of the amino acid by the target body upon oral administration. Without being bound by theory, bioavailability of amino acids may be enhanced by, for example, suppressing P-glycoproteins and/or modulating other physical barrier mechanisms that would otherwise reduce the penetration of amino acids, for example, into the epithelial cells or through the blood-brain barrier of a subject. Additionally or alternatively, bioavailability may be enhanced by protecting the amino acids from degradation either during storage or in vivo. Additionally or alternatively, bioavailability may be enhanced by improving the solubility or emulsifiability of amino acids because of its linkage to the sophorolipid structure, either in a composition prior to administration or in vivo after administration.

In other embodiments, the modified sophorolipids enhance the efficacy of the sophorolipid as well as the amino acid.

Depending on the nature and effects of the amino acid being carried in the modified sophorolipids of the present invention are able to treat or prevent various diseases, ailments, and/or conditions.

As used herein, the term “treatment” refers to eradicating, reducing, ameliorating, or reversing, a degree, sign or symptom of a condition or disorder to any extent, and includes, but does not require, a complete cure of the condition or disorder. Treating can be curing, improving, or partially ameliorating a disorder.

As used herein, “preventing” a condition or disorder refers to avoiding, delaying, forestalling, or minimizing the onset of a particular sign or symptom of the condition or disorder. Prevention can, but is not required to be, absolute or complete, meaning the sign or symptom may still develop at a later time. Prevention can include reducing the severity of the onset of such a condition or disorder, and/or inhibiting the progression of the condition or disorder to a more severe condition or disorder.

One aspect of the present invention relates to a method of reducing stress factors in a subject by orally administering to the subject a modified sophorolipid of the formula (I) and/or (II), or a salt thereof, wherein R₁ is methionine, lysine, and/or glutamine linked to the carbonyl moiety of the sophorolipid structure via an amide linkage. Stress factors may include cortisol levels, ROS levels, and/or biomarkers of oxidative stress known to those skilled in the art, such as those disclosed in Katerji M., Approaches and Methods to Measure Oxidative Stress in Clinical Samples: Research Applications in the Cancer Field, Oxid. Med. Cell. Longev. 2019 (2019).

Other aspects of the present invention relate to a method of treating or preventing a muscular disorder or disease in a subject by orally administering to the subject a modified sophorolipid of the formula (I) and/or (II), or a salt thereof, wherein R₁ is taurine linked to the carbonyl moiety of the sophorolipid structure via an amide linkage. The muscular disorder or disease may be, for example, disuse-induced muscle atrophy, muscular dystrophy and/or senescence, as well as sarcolemmal hyper-excitability in myotonia-related syndromes.

In some embodiments, the present invention provides a method of increasing muscular vitality in a subject by orally administering to the subject a modified sophorolipid of the formula (I) and/or (II), or a salt thereof, wherein R₁ is taurine linked to the carbonyl moiety of the sophorolipid structure via an amide linkage. Muscular vitality may be assessed by measuring the subject's muscular volume, muscular strength, muscular power, and/or muscular endurance.

Yet other aspects of the present invention relate to a method of treating or preventing a cardiovascular disorder or disease in a subject by orally administering to the subject a modified sophorolipid of the formula (I) and/or (II), or a salt thereof, wherein R₁ is taurine linked to the carbonyl moiety of the sophorolipid structure via an amide linkage. The cardiovascular disorder or disease may be, for example, atherosclerosis, hyperhomocysteinemia, cardiomyopathy, and coronary heart disease.

In some embodiments, the present invention provides a method of improving a subject's cardiovascular vitality by orally administering to the subject a modified sophorolipid of the formula (I) and/or (II), or a salt thereof, wherein R₁ is taurine linked to the carbonyl moiety of the sophorolipid structure via an amide linkage. Improvements may be observed in, for example, the subject's blood pressure, heart rate, and blood vessels.

Further aspects of the present invention relate to a method of treating or preventing hypertension in a subject by orally administering to the subject a modified sophorolipid of the formula (I) and/or (II), or a salt thereof, wherein R₁ is taurine linked to the carbonyl moiety of the sophorolipid structure via an amide linkage.

Other aspects of the present invention relate to a method of treating or preventing a disorder or disease of the central nervous system or peripheral nervous system in a subject by orally administering to the subject a modified sophorolipid of the formula (I) and/or (II), or a salt thereof, wherein R₁ is taurine linked to the carbonyl moiety of the sophorolipid structure via an amide linkage. The disorder or disease of the central nervous system or peripheral nervous system may be, for example, memory loss and Alzheimer's disease.

In some embodiments, the present invention provides a method of improving the function of the central nervous system or peripheral nervous system in a subject by orally administering to the subject a modified sophorolipid of the formula (I) and/or (II), or a salt thereof, wherein R₁ is taurine linked to the carbonyl moiety of the sophorolipid structure via an amide linkage. Improvements may be seen in, for example, the subject's memory retention or motor skills.

Additional aspects of the present invention relate to a method of treating or preventing a disorder or disease of the connective tissue in a subject by orally administering to the subject a modified sophorolipid of the formula (I) and/or (II), or a salt thereof, wherein R₁ is taurine linked to the carbonyl moiety of the sophorolipid structure via an amide linkage.

Other aspects of the present invention relate to a method of strengthening the connective tissue of a subject by orally administering to the subject a modified sophorolipid of the formula (I) and/or (II), wherein R₁ is taurine linked to the carbonyl moiety of the sophorolipid structure via an amide linkage.

Further embodiments of the present invention are directed to a method of improving nutrition in a subject's diet by administering the modified sophorolipid incorporating the amino acid(s) that is or may be lacking, or is insufficient in the subject's diet.

In some embodiments, the various methods according to the present invention require the modified sophorolipids of the formula (I) and/or (II), or a salt thereof, in a composition in the form of solids including tablets, filled capsules, and pellet forms. In another embodiment, the pharmaceutical composition may be in the powder form, in which the pharmaceutically accepted carrier is a finely divided solid that is in a mixture with the finely divided active ingredient, i.e., the modified sophorolipids. In a further embodiment, the pharmaceutical composition according to the present invention is a sustained release system such as semipermeable matrices of solid hydrophobic polymers containing the modified sophorolipids of the present invention. In another embodiment, the pharmaceutical composition is in a liquid form such as aqueous or non-aqueous solutions, suspensions, emulsions, elixirs, and capsules filled with the same.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention, e.g., the ability to improve the bioavailability of a substance. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example, within 2 standard deviations of the mean. As further examples, “about” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### EXAMPLES

Following are exemplary compounds of the present invention, which are offered by way of illustration and are not intended to limit the invention. The present invention covers all of the compounds represented by the general formulas (I) and (II), including salts thereof, hydrates thereof, geometric and optical isomers thereof, and polymorphic forms thereof. Various modifications or changes within the spirit and purview of this application will be suggested to persons skilled in the art.
Exemplary compounds of formula (I):

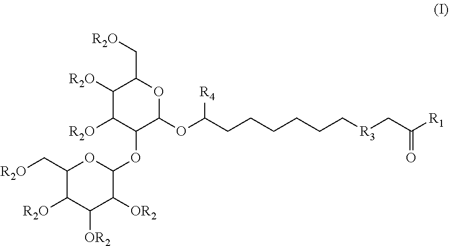

wherein R₂ is H, R₃ is an aliphatic hydrocarbon chain of 8 carbons with one degree of unsaturation in the form of a double bond, R₄ is methyl (—CH₃), and R₁ is as defined in Table 1.

TABLE 1
Compound numbers and corresponding R₁ group of formula (I)

| Compound | |
|---|---|
| Number | R₁ |
| 1 | 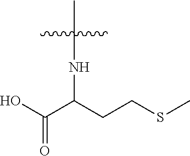 [SMILES: *NC(CCSC)C(=O)O] |
| | Methionine |
| 2 | 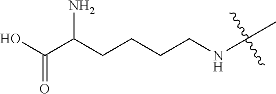 [SMILES: *NCCCCC(N)C(=O)O] |
| | Lysine |
| 3 | 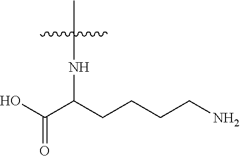 [SMILES: *NC(CCCCN)C(=O)O] |
| | Lysine |
| 4 | 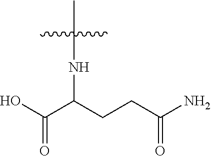 [SMILES: *NC(CCC(N)=O)C(=O)O] |
| | Glutamine |
| 5 | 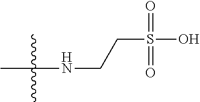 [SMILES: *NCCS(=O)(=O)O] |
| | Taurine |

Exemplary compounds of formula (II):

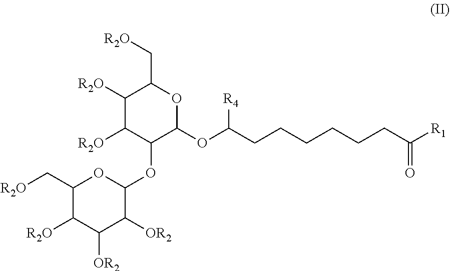

wherein R₂ is H, R₄ is methyl (—CH₃), and R₁ is as defined in Table 2.

TABLE 2
Compound numbers and corresponding R₁ group of formula (II)

| Compound | |
|---|---|
| Number | R₁ |
| 6 | 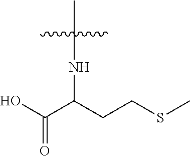 [SMILES: *NC(CCSC)C(=O)O] |
| | Methionine |
| 7 | 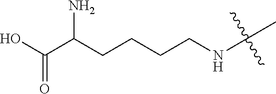 [SMILES: *NCCCCC(N)C(=O)O] |
| | Lysine |
| 8 | 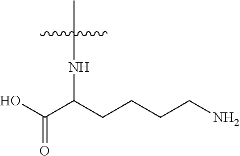 [SMILES: *NC(CCCCN)C(=O)O] |
| | Lysine |
| 9 | 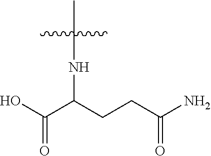 [SMILES: *NC(CCC(N)=O)C(=O)O] |
| | Glutamine |
| 10 | 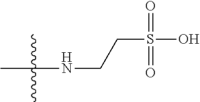 [SMILES: *NCCS(=O)(=O)O] |
| | Taurine |

1. A compound of formula (I) or a salt thereof: 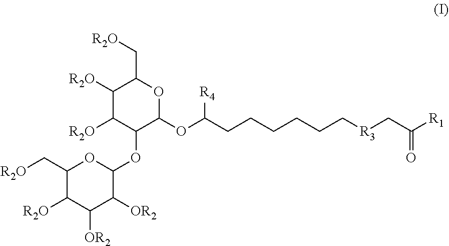
wherein:
R₁ is an amino acid linked to the adjacent carbonyl moiety via an amide linkage;
R₂ is each independently H or —COCH₃;
R₃ is a saturated or unsaturated aliphatic hydrocarbon chain having 5-15 carbons; and
R₄ is H or —CH₃.

2. The compound of claim 1, wherein the amino acid is taurine.

3. The compound of claim 1, wherein the amino acid is methionine, lysine, or glutamine.

4. The compound of claim 1, wherein the compound is a sodium (Na⁺) or potassium (K⁺) salt of the amino acid.

5. A compound of formula (II) or a salt thereof 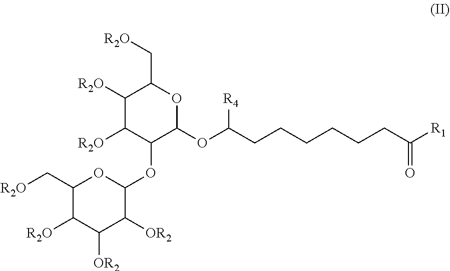
wherein:
R₁ is an amino acid linked to the adjacent carbonyl moiety via an amide linkage;
R₂ is each independently H or —COCH₃; and
R₄ is H or —CH₃.

6. The compound of claim 5, wherein the amino acid is taurine.

7. The compound of claim 5, wherein the amino acid is methionine, lysine, or glutamine.

8. The compound of claim 5, wherein the compound is a sodium (Na⁺) or potassium (K⁺) salt of the amino acid.

9. A composition comprising the compound of claim 1 and a pharmaceutically or cosmetically acceptable carrier.

10. The composition of claim 9, further comprising the compound of claim 5.

11. A composition comprising the compound of claim 5 and a pharmaceutically or cosmetically acceptable carrier.

12. A method of improving the oral bioavailability of an amino acid in a subject comprising orally administering to the subject a modified sophorolipid of formula (I) and/or (II), or a salt thereof, 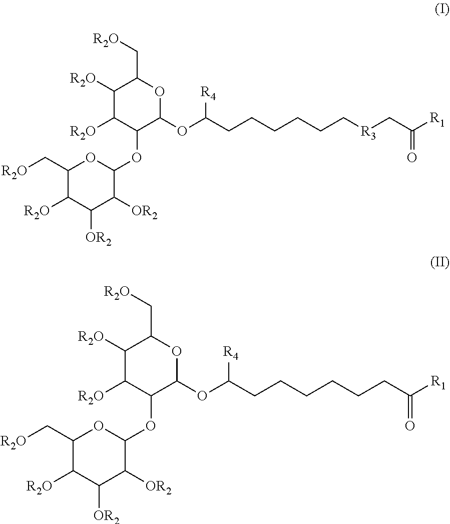
wherein R₁ is the amino acid linked to the adjacent carbonyl moiety via an amide linkage, R₂ is each independently H or —COCH₃, R₃ is a saturated or unsaturated aliphatic hydrocarbon chain having 5-15 carbons, and R₄ is each independently H or —CH₃.

13. The method of claim 12, wherein the amino acid is taurine, methionine, lysine, or glutamine.

14. A method of treating or preventing a disease, disorder, or condition in a subject comprising administering to the subject a modified sophorolipid of the formula (I) and/or (II) or a salt thereof: 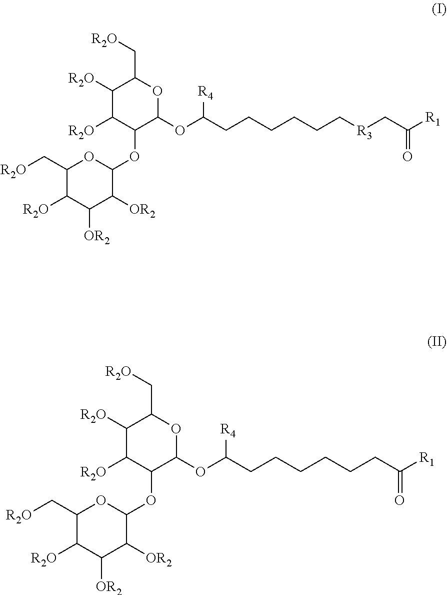
wherein R₁ is each independently an amino acid linked to the adjacent carbonyl moiety via an amide linkage, R₂ is each independently H or —COCH₃, R₃ is a saturated or unsaturated aliphatic hydrocarbon chain having 5-15 carbons, and R₄ is each independently H or —CH₃.

15. The method of claim 14, wherein said disease, disorder, or condition is increased stress factors, and wherein the amino acid is methionine, lysine, and/or glutamine.

16. The method of claim 14, wherein said disease, disorder, or condition is a muscular disorder or disease, and wherein said amino acid is taurine.

17. The method of claim 14, wherein said disease, disorder, or condition is a cardiovascular disorder or disease, and wherein said amino acid is taurine.

18. The method of claim 14, wherein said disease, disorder, or condition is hypertension, and wherein said amino acid is taurine.

19. The method of claim 14, wherein said disease, disorder, or condition is a disorder or disease of the central nervous system or peripheral nervous system, and wherein said amino acid is taurine.

20. The method of claim 14, wherein said disease, disorder, or condition is a disorder or disease of the connective tissue, and
wherein said amino acid is taurine.

21. A method of improving muscular vitality in a subject comprising administering to the subject in need of such improvement a modified sophorolipid of the formula (I) and/or (II) or a salt thereof: 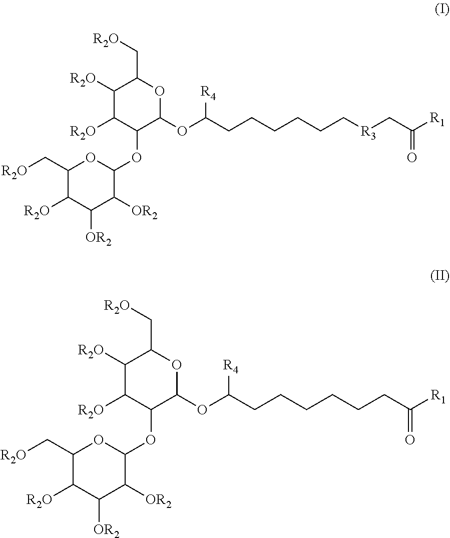
wherein R₁ is an amino acid linked to the adjacent carbonyl moiety via an amide linkage, R₂ is each independently H or —COCH₃, R₃ is a saturated or unsaturated aliphatic hydrocarbon chain having 5-15 carbons, and R₄ is each independently H or —CH₃; and
wherein said amino acid is taurine.
